(19) United States
(12) Patent Application Publication
(10) Pub. No.: US 2011/0081607 A1
(43) Pub. Date: Apr. 7, 2011

(54) METHYLOL COMPOUND, ALDEHYDE COMPOUND, METHOD FOR PREPARING THE METHYLOL COMPOUND USING THE ALDEHYDE COMPOUND, AND PHOTORECEPTOR USING THE METHYLOL COMPOUND

(75) Inventors: Yuuji Tanaka, Numazu-shi (JP); Norio Nagayama, Numazu-shi (JP)

(73) Assignee: RICOH COMPANY, LTD.,, Tokyo (JP)

(21) Appl. No.: 12/887,652

(22) Filed: Sep. 22, 2010

(30) Foreign Application Priority Data
Oct. 2, 2009 (JP) 2009-230220

(51) Int. Cl. G03G 5/047; C07C 215/68; C07C 211/54

(52) U.S. Cl. 430/58.75; 564/443; 564/305

(57) ABSTRACT

A methylol compound having a specific formula such that two triphenylamine groups each having two methylol groups are connected with each other by an oxygen atom, a methylene group, a vinylene group, or an ethylene group. The methylol compound is preferably prepared by subjecting a corresponding aldehyde compound to a reduction reaction in the presence of a reducing agent. The methylol compound is preferably used as a charge transport material.

### BACKGROUND OF THE INVENTION

1. Field of the Invention

The present invention relates to a methylol compound for use as an organic charge transport material and a photoconductor, and to a method for preparing the methylol compound. In addition, the present invention also relates to an aldehyde compound for use as a raw material for the methylol compound. Further, the present invention relates to a photoreceptor using the methylol compound.

2. Description of the Related Art

Organic semiconductor materials having a charge transport function are preferably used as film forming materials for use in organic devices such as organic electrophotographic photoreceptors, organic electroluminescent devices, organic thin film transistors, and organic solar cells.

In order to impart a charge transport function to a resin, a method in which a charge transport material is dispersed in a binder resin capable of forming a film is typically used for preparing an organic device such as electrophotographic photoreceptors.

However, such a charge transport film cannot have good characteristics (e.g., a good combination of mechanical strength and heat resistance). Therefore, in order that the charge transport film have good characteristics, it is effective to integrate a charge transport material with a binder resin. In this regard, in attempting to improve the mechanical strength (i.e., abrasion resistance) of a photosensitive layer of an electrophotographic photoreceptor to prolong the life of the photoreceptor, various proposals have been made.

For example, one approach involves a crosslinkable silicone resin including a colloidal silica used for forming an outermost layer of a photoreceptor. However, the electrophotographic properties of the resultant photoreceptor deteriorate after long repeated use, forming defective images such as images having background development and blurred images. Namely, the photoreceptor has insufficient durability to be used as a long-life photoreceptor, which is needed recently.

There is another proposal such that a resin layer in which an organic silicon-modified positive hole transport compound is bonded with a crosslinked organic silicone polymer is used as an outermost layer of a photoreceptor. However, the photoreceptor of ten produces blurred images. In order to prevent formation of such blurred images, a heater for heating the photoreceptor has to be provided in an image forming apparatus, resulting in increase in size and costs of the image forming apparatus. In addition, since the potential of an irradiated portion (i.e., residual potential) of the photoreceptor is relatively high, the photoreceptor produces low-density images particularly when using a low-potential developing process in which the potential of a charged photoreceptor is kept relatively low.

There is another proposal for a photoreceptor including a resin layer having a three-dimensional network formed by crosslinking a crosslinkable siloxane resin including a charge transport group. However, the photoreceptor often causes a cracking problem in that a crack is formed in the resin layer due to contraction of the layer particularly when the silicone resin is used in combination with a commercially available coating agent having low costs and good handling property. In addition, since the residual potential of the photoreceptor increases depending on the thickness of the resin layer, the photoreceptor produces images when using a low-potential developing process. Further, when the number of charge transport groups is increased, the mechanical strength of the resultant resin layer decreases, adversely affecting durability of the photoreceptor, and the photoreceptor often forms blurred images. Therefore, it is difficult for the technique to produce a photoreceptor that can repeatedly produce images of good quality over a long period of time at low cost.

Further, there is another proposal for a photoreceptor having a protective layer including a charge transport material having at least one hydroxyl group, a three-dimensionally crosslinked resin, and a particulate electroconductive material. However, including a particulate electroconductive material in a protective layer decreases the volume resistance of the layer, resulting in formation of blurred images particularly under high temperature and high humidity conditions due to blurring of electrostatic latent images corresponding to the images.

Furthermore, there is a proposal for a photoreceptor having a protective layer including a crosslinked resin formed by subjecting a polyol having a reactive charge transport group and at least two hydroxyl groups, and an aromatic isocyanate compound to a crosslinking reaction. The photoreceptor has good durability and can produce high-quality images at a high speed over a long period of time without forming abnormal images such as low-density images. However, in order to further reduce environmental burdens, development of a new compound is desired to prepare a photoreceptor having better durability, i.e., a longer life.

For these reasons, there is a need for a compound that has good compatibility with other monomers and polymers for use in preparing a layer and can form a layer having a good combination of mechanical strength (such as abrasion resistance and cracking resistance) and heat resistance while imparting a good charge transport property to the resultant photoreceptor.

### SUMMARY

This patent specification describes a novel methylol compound having the following formula (1):

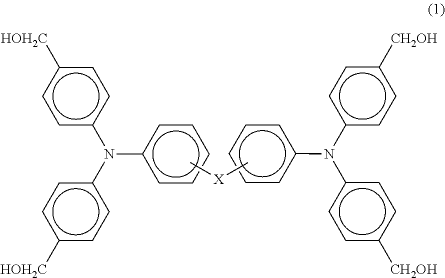

wherein X represents a group —O—, —CH₂—, —CH═CH— or —CH₂CH₂—.

This patent specification further describes a novel aldehyde compound having the following formula (2):

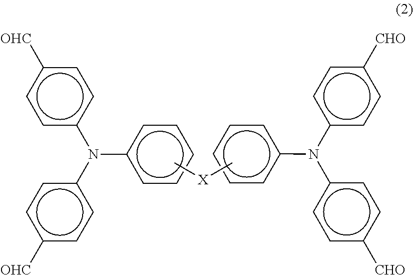

wherein X is defined above.

This patent specification further describes a novel method for forming a methylol compound having formula (1) including subjecting a compound having formula (2) to a reduction reaction in the presence of a reducing agent.

This patent specification further describes a novel charge transport layer including a compound having formula (1) and a binder resin.

This patent specification further describes a novel photoreceptor including a substrate, an optional undercoat layer located on the substrate, a charge generation layer, and the above-mentioned charge transport layer, wherein the charge generation layer and the charge transport layer are overlaid on the substrate or the optional undercoat layer.

### BRIEF DESCRIPTION OF THE DRAWINGS

A more complete appreciation of aspects of the invention and many of the attendant advantage thereof will be readily obtained as the same become better understood by reference to the following detailed description when considered in connection with the accompanying drawings, wherein:

FIG. 1 is an infrared absorption spectrum of a triphenyl amine compound prepared in Synthesis Example 1;

FIG. 2 is an infrared absorption spectrum of an aldehyde compound of the present invention prepared in Synthesis Example 2;

FIG. 3 is an infrared absorption spectrum of a methylol compound of the present invention prepared in Synthesis Example 3;

FIG. 4 is an infrared absorption spectrum of a triphenyl amine compound prepared in Synthesis Example 4;

FIG. 5 is an infrared absorption spectrum of an aldehyde compound of the present invention prepared in Synthesis Example 5;

FIG. 6 is an infrared absorption spectrum of a methylol compound of the present invention prepared in Synthesis Example 6;

FIG. 7 is an infrared absorption spectrum of a triphenyl amine compound prepared in Synthesis Example 7;

FIG. 8 is an infrared absorption spectrum of an aldehyde compound of the present invention prepared in Synthesis Example 8;

FIG. 9 is an infrared absorption spectrum of a methylol compound of the present invention prepared in Synthesis Example 9;

FIG. 10 is an infrared absorption spectrum of a triphenyl amine compound prepared in Synthesis Example 10;

FIG. 11 is an infrared absorption spectrum of an aldehyde compound of the present invention prepared in Synthesis Example 11; and

FIG. 12 is an infrared absorption spectrum of a methylol compound of the present invention prepared in Synthesis Example 12.

### DESCRIPTION OF THE PREFERRED EMBODIMENTS

It will be understood that if a layer is referred to as being “on” another layer, then it can be directly on the other layer, or intervening layers may be present.

The methylol compound of the present invention has the following formula (1):

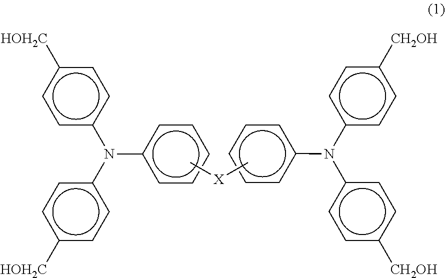

wherein X represents a group —O—, —CH₂—, —CH═CH— and —CH₂CH₂—.

The compound has a good charge transport function and can be used as an organic semiconductor material for forming electronic devices such as electrophotographic photoreceptors, organic electroluminescent devices, organic thin film transistors, and organic solar cells.

In this application, methylol compounds are defined as compounds having a methylol group (i.e., a methyl alcohol group). Specific examples of the methylol compound of the present invention having formula (1) include the following compounds listed in Table 1 below, wherein only the group —Ar—X—Ar— of each of the methylol compounds is described therein. However, the methylol compound of the present invention is not limited thereto.

| | |
|---|---|
| Methylol compound | 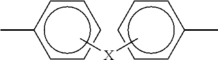 |
| Methylol compound 1 | 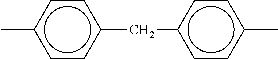 |
| Methylol compound 2 | 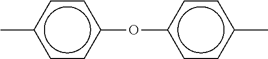 |
| Methylol compound 3 | 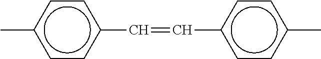 |
| Methylol compound 4 | 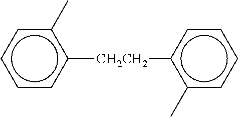 |
| Methylol compound 5 | 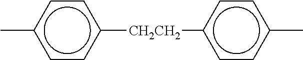 |
| Methylol compound 6 | 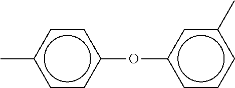 |

Methylol compounds of the present invention having formula (1) are novel compounds, and can be synthesized by subjecting an aldehyde compound having formula (2) to a reduction reaction in the presence of a reducing agent such as sodium borohydride (i.e., sodium tetrahydroborate). For example, such methylol compounds can be prepared by synthesizing an aldehyde compound having formula (2) according to the below-mentioned procedure, and then subjecting the aldehyde compound to a reduction reaction.

For example, an aldehyde compound having formula (2) can be synthesized as illustrated by the following reaction formula.

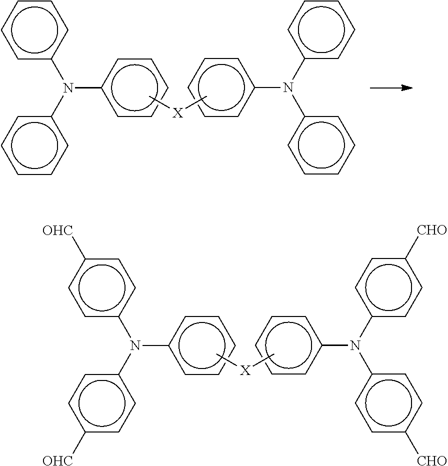

Specifically, a triphenyl amine compound is formylated by a known method such as Vilsmeier-Haack reaction to form an aldehyde compound having formula (2).

More specifically, formylation is preferably performed using a combination of zinc chloride, phosphorus oxychloride, and dimethylformamide. However, the method for synthesizing an aldehyde compound having formula (2) (i.e., an intermediate or a raw material for a methylol compound having formula (1)) is not limited thereto. Specific examples of the synthesizing method will be described later.

For example, a methylol compound having formula (1) of the present invention can be synthesized as illustrated by the following reaction formula.

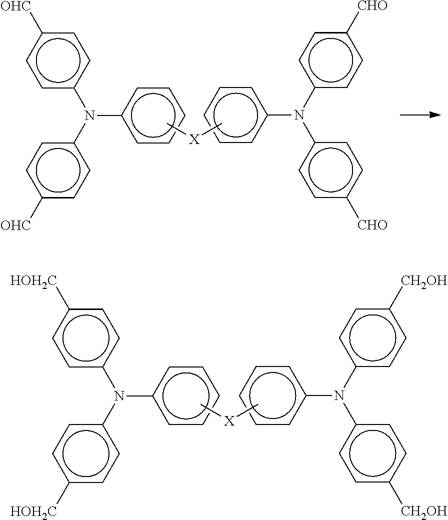

Specifically, the above-mentioned reduction reaction is preferably performed using sodium borohydride. However, the method for synthesizing a methylol compound having formula (1) is not limited thereto. Specific examples of the synthesizing method will be described later.

Since methylol compounds having formula (1) have a main skeleton such that two triphenyl amine groups are connected with each other with a group therebetween in a molecule, the compounds have a charge transport function and low crystallinity while having good compatibility with other monomers and polymers such as polycarbonate. In addition, the methylol compounds can easily react with a hydroxyl group of an isocyanate compound, thereby forming a crosslinked film having a high crosslinking degree while having a good charge transport property. Therefore, the methylol compounds of the present invention can be preferably used as organic functional materials for use in preparing various organic semiconductor devices such as organic electrophotographic photoreceptors, organic electroluminescent devices, organic thin film transistors, and organic solar cells.

Methylol compounds having formula (1) have good compatibility with other monomers and polymers (such as polycarbonate). Specific examples of such monomers include trimethylolpropane, butane triol, trimethylolpropane acrylate, hexamethylene diisocyanate, isophorone diisocyanate, SUMIDUR HT (registered trademark) (fromSumika Bayer Urethane Co., Ltd.), aromatic isocyanate compounds such as tolylene diisocyanate (TDI), diphenylmethane diisocyanate (MDI), polymeric MDI, xylene diisocyanate (XDI), and adducts of TDI, MDI, XDI with a polyol such as trimethylol propane, etc.

One or more of such monomers can be mixed with a methylol compound having formula (1) so that the resultant film has desired properties. The weight ratio (Mo/Me) of a monomer to a methylol compound, which is determined depending on the desired properties of the resultant film, is typically from 0.0001 to 15 (i.e., 0.01% to 1500%), and preferably from 0.01 to 5 (i.e., 1% to 500%) when the film is used for a charge transport layer of an electrophotographic photoreceptor.

When an aromatic isocyanate compound is used, the content of isocyanate groups in a molecule of the aromatic isocyanate compound, i.e., the weight ratio ([NCO]/[isocyanate compound]) of isocyanate groups to the isocyanate compound, is preferably from 0.03 to 0.50 (i.e., 3% to 50%), and more preferably from 0.1 to 0.5 (i.e., 10% to 50%).

Having generally described this invention, further understanding can be obtained by reference to certain specific examples which are provided herein for the purpose of illustration only and are not intended to be limiting. In the descriptions in the following examples, the numbers represent weight ratios in parts unless otherwise specified.

### EXAMPLES

### Synthesis Example 1

The methylol compound 1 listed in Table 1 was prepared as follows.

### (1) Synthesis of Raw Material for Aldehyde Compound Used as Intermediate for Methylol Compound 1

The procedure for synthesizing the raw material is illustrated by the following reaction formula.

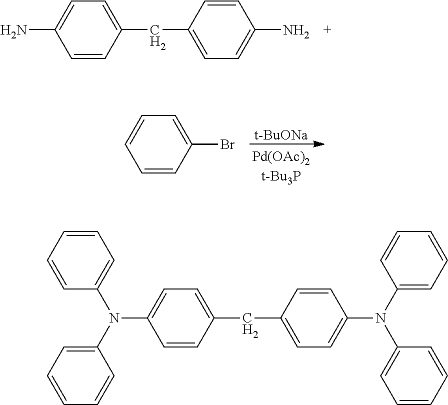

Specifically, the following components were fed into a four-necked flask.

| | | |
|---|---|---|
| 4,4′-Diaminodiphenyl methane | 19.83 | g |
| Bromobenzene | 69.08 | g |
| Palladium acetate | 2.24 | g |
| Sodium t-butoxide | 46.13 | g |
| Ortho-xylene | 250 | ml |

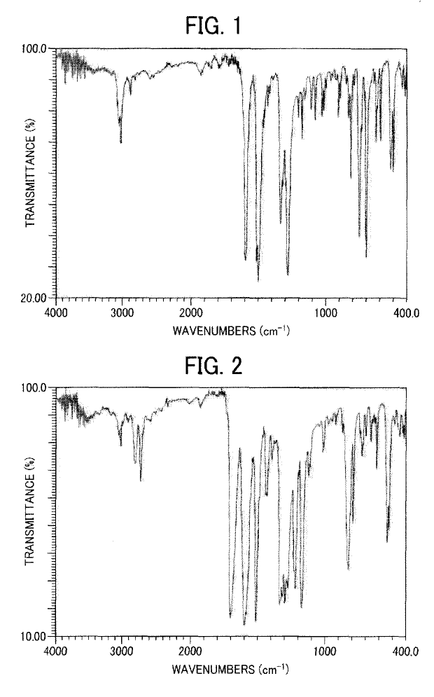

The components were mixed by an agitator at room temperature under an argon gas flow. Next, 8.09 g of tri-t-butyl phosphine was dropped into the flask while agitating. After the mixture was heated to 80° C. while agitated, the mixture was further agitated for 1 hour while refluxed. Further, the mixture was diluted with toluene, and magnesium sulfate, activated earth, and silica gel were added to the reaction product, followed by filtering, washing and condensing, resulting in preparation of a crystalline material. The crystalline material was dispersed in methanol, followed by filtering, washing and drying. Thus, 45.73g of a pale yellow powder (hereinafter referred to as a raw material 1) was prepared. The infrared absorption spectrum of the raw material 1 is illustrated in FIG. 1.

### (2) Synthesis of Intermediate (Aldehyde Compound) for Compound 1

The procedure for synthesizing the intermediate (i.e., aldehyde compound) for the compound 1 is illustrated by the following reaction formula.

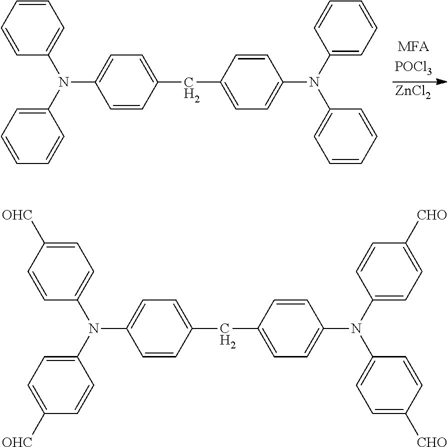

Specifically, the following components were fed into a four-necked flask.

| | | |
|---|---|---|
| Raw material 1 prepared above | 30.16 | g |
| N-Methylformanilide | 71.36 | g |
| Ortho-dichlorobenzene | 400 | ml |

The components were mixed by an agitator at room temperature under an argon gas flow. Next, 82.01 g of phosphorus oxychloride was dropped into the flask. After the mixture was heated to 80° C. while agitated, 32.71 g of zinc chloride was dropped thereinto, and the mixture was agitated for about 10 hours at 80° C., followed by agitating at 120° C. Next, an aqueous solution of potassium hydroxide was added thereto to perform a hydrolysis reaction. The reaction product was extracted using dichloromethane, and the extracted material was subjected to a dehydration treatment using magnesium sulfate, followed by an absorption treatment using activated earth, filtering, washing and condensing, resulting in preparation of a crystalline material. The crystalline material was subjected to a refining treatment to be isolated using a silica gel column and a mixture solvent of toluene and ethyl acetate mixed at a weight ratio of 8/2. The thus-prepared crystalline material was subjected to a re-crystallization treatment using a mixture solvent of methanol and ethyl acetate. Thus, 27.80 g of a yellow powder (hereinafter referred to as an aldehyde compound 1) was prepared. The infrared absorption spectrum of the aldehyde compound 1 is illustrated in FIG. 2.

### (3) Synthesis of the Methylol Compound 1

The procedure for synthesizing the methylol compound 1 is illustrated by the following reaction formula.

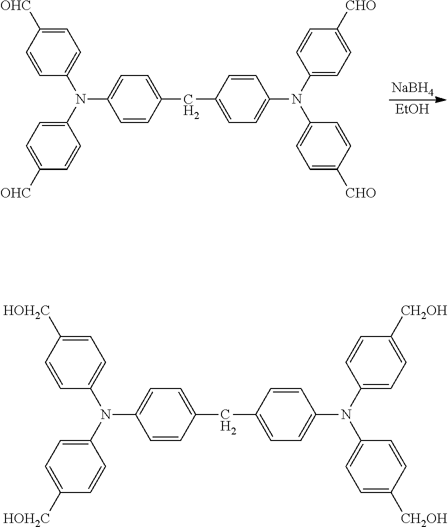

Specifically, the following components were fed into a four-necked flask.

| | | |
|---|---|---|
| Aldehyde compound 1 prepared above | 12.30 | g |
| Ethanol | 150 | ml |

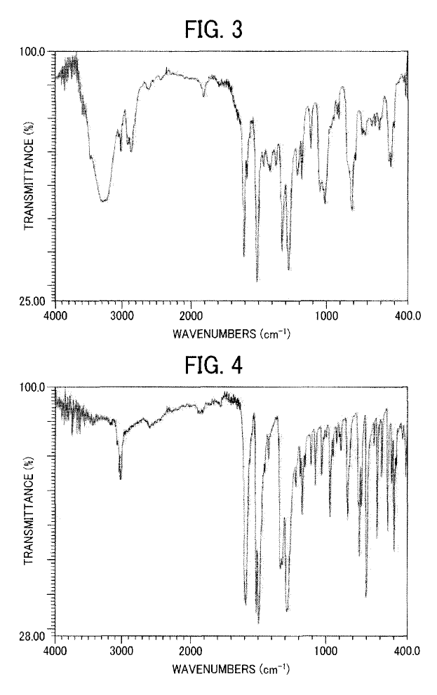

The components were mixed by an agitator at room temperature. Next, 3.63 g of sodium borohydride was added thereto, and the mixture was agitated for 4 hours. The reaction product was extracted using ethyl acetate, and the extracted material was subjected to a dehydration treatment using magnesium sulfate, followed by an absorption treatment using activated earth and silica gel, filtering, washing and condensing, resulting in preparation of an amorphous material. The amorphous material was dispersed in n-hexane, followed by filtering, washing and drying. Thus, 12.0 g of a pale yellowish white amorphous material (i.e., methylol compound 1) was prepared. The infrared absorption spectrum of the methylol compound 1 is illustrated in FIG. 3.

### Synthesis Example 2

The methylol compound 2 listed in Table 1 was prepared as follows.

### (1) Synthesis of Raw Material for Aldehyde Compound Used as Intermediate for Methylol Compound 2

The procedure for synthesizing the raw material is illustrated by the following reaction formula.

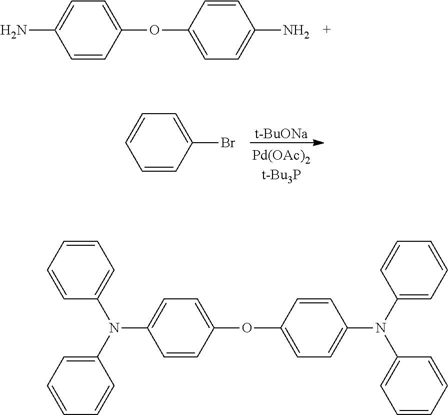

Specifically, the following components were fed into a four-necked flask.

| | | |
|---|---|---|
| 4,4′-Diaminodiphenyl ether | 20.02 | g |
| Bromobenzene | 69.08 | g |
| Palladium acetate | 0.56 | g |
| Sodium t-butoxide | 46.13 | g |
| Ortho-xylene | 250 | ml |

The components were mixed by an agitator at room temperature under an argon gas flow. Next, 2.02 g of tri-t-butyl phosphine was dropped into the flask while agitating. After the mixture was heated to 80° C. while agitated, the mixture was further agitated for 1 hour while refluxed. Further, the reaction product was diluted with toluene, and magnesium sulfate, activated earth, and silica gel were added thereto, followed by agitating, filtering, washing and condensing, resulting in preparation of a crystalline material. The crystalline material was dispersed in methanol, followed by filtering, washing and drying. Thus, 43.13 g of a pale brown powder (hereinafter referred to as a raw material 2) was prepared. The infrared absorption spectrum of the raw material 2 is illustrated in FIG. 4.

### (2) Synthesis of Intermediate (Aldehyde Compound) for Methylol Compound 2

The procedure for synthesizing the intermediate (i.e., aldehyde compound) for the methylol compound 2 is illustrated by the following reaction formula.

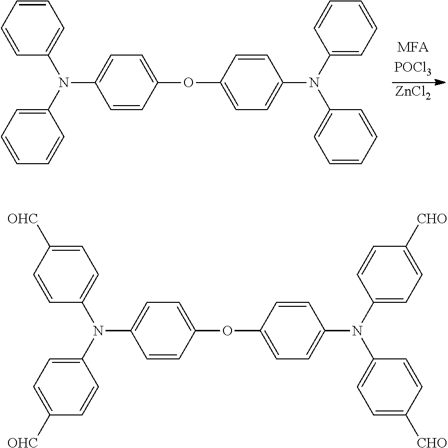

Specifically, the following components were fed into a four-necked flask.

| | | |
|---|---|---|
| Raw material 2 prepared above | 30.27 | g |
| N-Methylformanilide | 71.36 | g |
| Ortho-dichlorobenzene | 300 | ml |

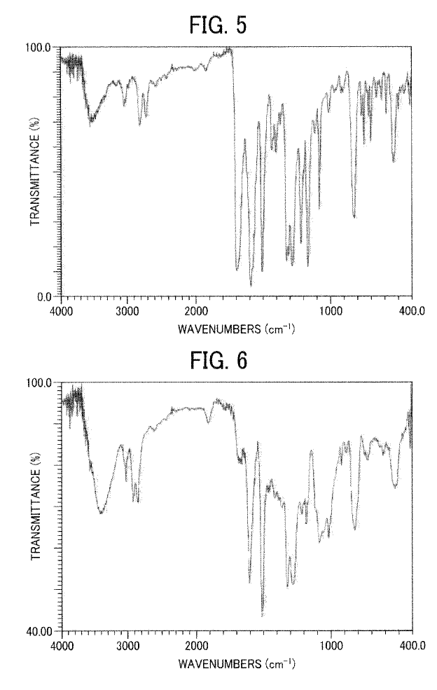

The components were mixed by an agitator at room temperature under an argon gas flow. Next, 82.01 g of phosphorus oxychloride was dropped into the flask. After the mixture was heated to 80° C. while agitated, 16.36 g of zinc chloride was dropped thereinto, and the mixture was agitated for 1 hour at 80° C., followed by agitating for 4 hours at 120° C., and agitating for 3 hours at 140° C. Next, an aqueous solution of potassium hydroxide was added thereto to perform a hydrolysis reaction. The reaction product was extracted using toluene, and the extracted material was subjected to a dehydration treatment using magnesium sulfate, followed by an absorption treatment using activated earth, filtering, washing and condensing. The resultant product was subjected to a refining treatment using a silica gel column and a mixture solvent of toluene and ethyl acetate, followed by condensing, resulting in preparation of a crystalline material. The crystalline material was dispersed in methanol, followed by filtering, washing and drying to prepare the target material. Thus, 14.17 g of a pale yellow powder (hereinafter referred to as an aldehyde compound 2) was prepared. The infrared absorption spectrum of the aldehyde compound 2 is illustrated in FIG. 5.

### (3) Synthesis of Methylol Compound 2

The procedure for synthesizing the methylol compound 2 is illustrated by the following reaction formula.

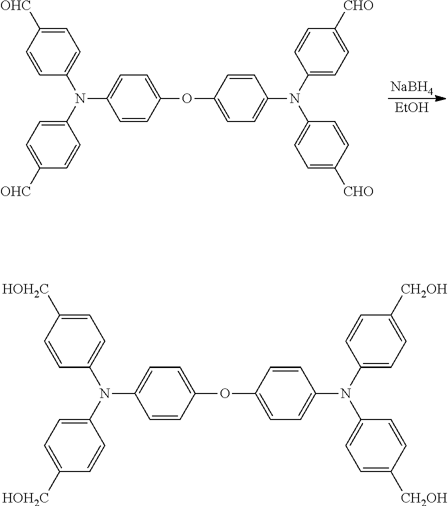

Specifically, the following components were fed into a four-necked flask.

| | | |
|---|---|---|
| Aldehyde compound 2 prepared above | 6.14 | g |
| Ethanol | 75 | ml |

The components were mixed by an agitator at room temperature. Next, 1.82 g of sodium borohydride was added thereto, and the mixture was agitated for 7 hours. The reaction product was extracted using ethyl acetate, and the extracted material was subjected to a dehydration treatment using magnesium sulfate, followed by an absorption treatment using activated earth and silica gel, filtering, washing and condensing, resulting in preparation of an amorphous material. The amorphous material was dispersed in n-hexane, followed by filtering, washing and drying. Thus, 5.25 g of a white amorphous material (i.e., methylol compound 2) was prepared. The infrared absorption spectrum of the methylol compound 2 is illustrated in FIG. 6.

### Synthesis Example 3

The methylol compound 3 listed in Table 1 was prepared.

### (1) Synthesis of Raw Material for Aldehyde Compound Used as Intermediate for Methylol Compound 3

The procedure for synthesizing the raw material is illustrated by the following reaction formula.

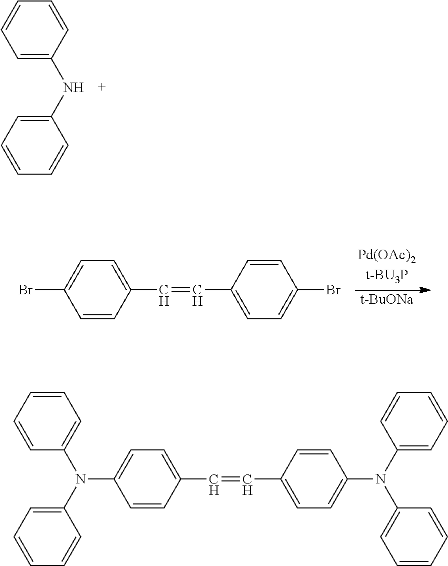

Specifically, the following components were fed into a four-necked flask.

| | | |
|---|---|---|
| Diphenylamine | 22.33 | g |
| Dibromostilbene | 20.28 | g |
| Palladium acetate | 0.336 | g |
| Sodium t-butoxide | 13.84 | g |
| Ortho-xylene | 150 | ml |

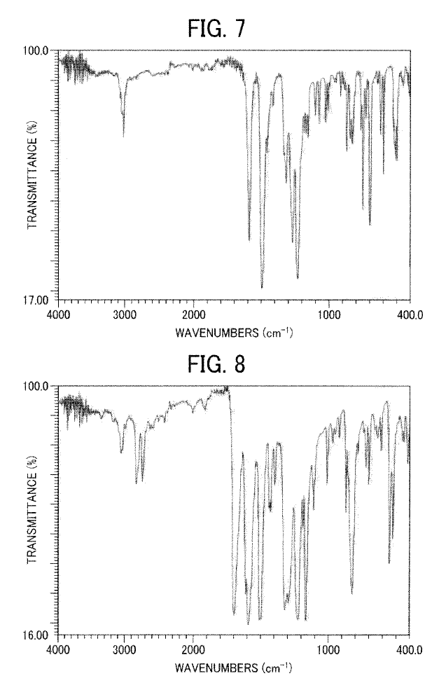

The components were mixed by an agitator at room temperature under an argon gas flow. Next, 1.22 g of tri-t-butyl phosphine was dropped into the flask while agitating. After the mixture was heated for 1 hour at 80° C. while agitated, the mixture was further agitated for 2 hours while refluxed. Further, the reaction product was diluted with toluene, and magnesium sulfate, activated earth, and silica gel were added thereto, followed by agitating, filtering, washing and condensing, resulting in preparation of a crystalline material. The crystalline material was dispersed in methanol, followed by filtering, washing and drying. Thus, 29.7 g of a pale yellow powder (hereinafter referred to as a raw material 3) was prepared. The infrared absorption spectrum of the raw material 3 is illustrated in FIG. 7.

### (2) Synthesis of Intermediate (Aldehyde Compound) for Methylol Compound 3

The procedure for synthesizing the intermediate (i.e., aldehyde compound) for the methylol compound 3 is illustrated by the following reaction formula.

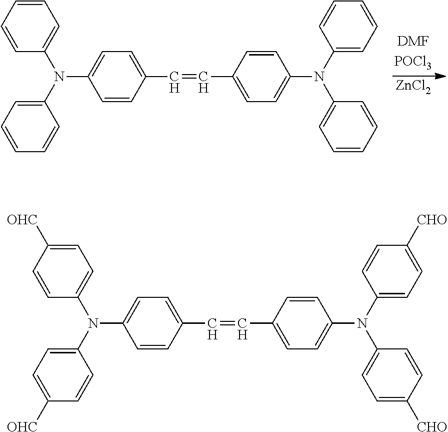

Specifically, the following components were fed into a four-necked flask.

| | |
|---|---|
| Dehydrated dimethylformaldehyde | 33.44 g |
| Dehydrated toluene | 84.53 g |

The mixture was agitated under an argon gas flow while cooled with ice water. Next, 63.8 g of phosphorus oxychloride was gradually dropped into the flask while agitating, and the mixture was further agitated for about 1 hour. A mixture of 26.76 g of the raw material 3 and 106 g of dehydrated toluene was gradually dropped into the flask. The mixture agitated for 1 hour at 80° C., followed by agitating for 5 hours while refluxed. Next, an aqueous solution of potassium hydroxide was added thereto to perform a hydrolysis reaction. After the reaction product was extracted using toluene, the extracted material was subjected to a dehydration treatment using magnesium sulfate, followed by a column refining treatment using a mixture solvent of toluene and ethyl acetate mixed at a weight ratio of 8/2 to be isolated. The thus prepared material was dispersed in methanol, followed by filtering, washing and drying. Thus, 16.66 g of an orange powder (hereinafter referred to as an aldehyde compound 3) was prepared. The infrared absorption spectrum of the aldehyde compound 3 is illustrated in FIG. 8.

### (3) Synthesis of Methylol Compound 3

The procedure for synthesizing the compound 3 is illustrated by the following reaction formula.

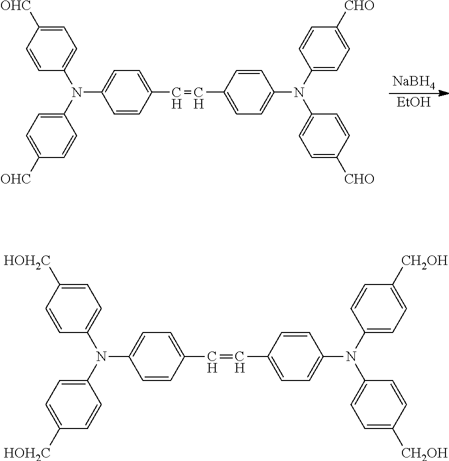

Specifically, the following components were fed into a four-necked flask.

| | | |
|---|---|---|
| Aldehyde compound 3 prepared above | 6.54 | g |
| Ethanol | 75 | ml |

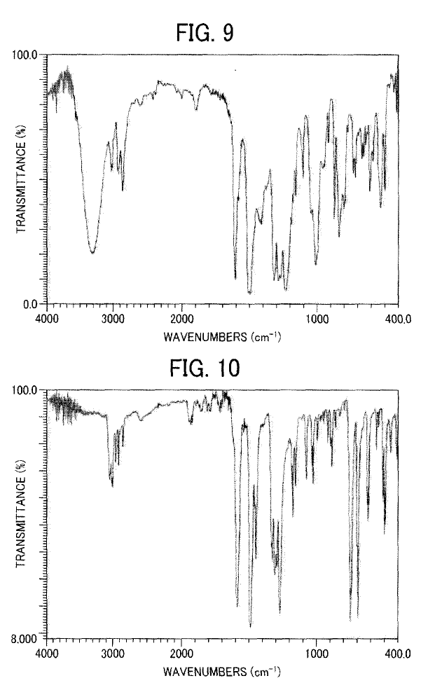

The components were mixed by an agitator at room temperature. Next, 1.82 g of sodium borohydride was added thereto, and the mixture was agitated for 4 hours. The reaction product was extracted using ethyl acetate, and the extracted material was subjected to a dehydration treatment using magnesium sulfate, followed by an absorption treatment using activated earth and silica gel, filtering, washing and condensing, resulting in preparation of an amorphous material. The amorphous material was dispersed in n-hexane, followed by filtering, washing and drying. Thus, 2.30 g of a yellow amorphous material (i.e., methylol compound 3) was prepared. The infrared absorption spectrum of the methylol compound 3 is illustrated in FIG. 9.

### Synthesis Example 4

The methylol compound 4 listed in Table 1 was prepared as follows.

### (1) Synthesis of Raw Material for Aldehyde Compound Used as Intermediate for Methylol Compound 4

The procedure for synthesizing the raw material is illustrated by the following reaction formula.

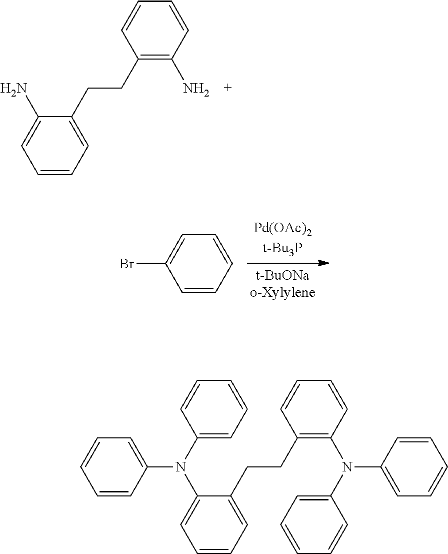

Specifically, the following components were fed into a four-necked flask.

| | | |
|---|---|---|
| 2,2′-Ethylenedianiline | 21.23 | g |
| Bromobenzene | 75.36 | g |
| Palladium acetate | 0.56 | g |
| Sodium t-butoxide | 46.13 | g |
| Ortho-xylene | 250 | ml |

The components were mixed by an agitator at room temperature under an argon gas flow. Next, 2.03 g of tri-t-butyl phosphine was dropped into the flask while agitated, and the mixture was agitated for 8 hours while refluxed. After the reaction product was diluted with toluene, magnesium sulfate, activated earth, and silica gel were added thereto, followed by agitating, filtering, washing and condensing, resulting in preparation of a crystalline material. The crystalline material was dispersed in methanol, followed by filtering, washing and drying. Thus, 47.65 g of a pale brown powder (hereinafter referred to as a raw material 4) was prepared. The infrared absorption spectrum of the raw material 4 is illustrated in FIG. 10.

### (2) Synthesis of Intermediate (Aldehyde Compound) for Methylol Compound 4

The procedure for synthesizing the intermediate (i.e., aldehyde compound) for the methylol compound 4 is illustrated by the following reaction formula.

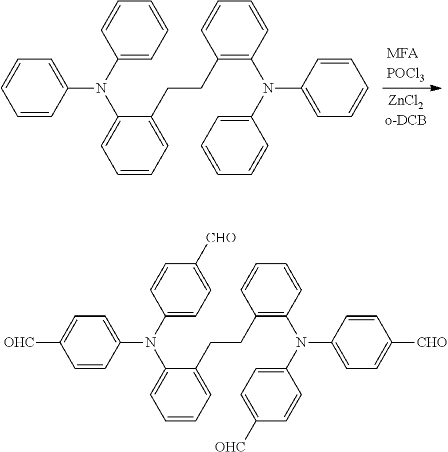

Specifically, the following components were fed into a four-necked flask.

| | | |
|---|---|---|
| Raw material 4 prepared above | 31.0 | g |
| N-Methylformanilide | 71.36 | g |
| Ortho-dichlorobenzene | 400 | ml |

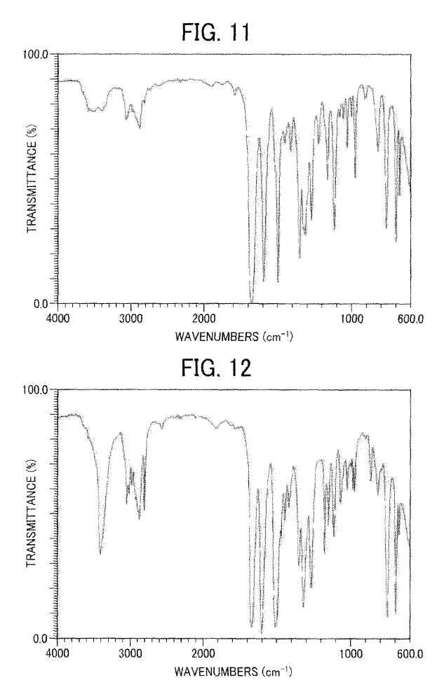

The components were mixed by an agitator at room temperature under an argon gas flow. Next, 82.01 g of phosphorus oxychloride was gradually dropped into the flask. After the mixture was heated to 80° C. while agitated, 32.71 g of zinc chloride was dropped thereinto, and the mixture was agitated for 1 hour at 80° C., followed by agitating for about 24 hours at 120° C. Next, an aqueous solution of potassium hydroxide was added thereto to perform a hydrolysis reaction. The reaction product was diluted with toluene, followed by washing with water. The oil phase liquid thereof was dehydrated using magnesium chloride, followed by an absorption treatment using activated earth, filtering, washing and condensing. Thus, 22.33 g of a yellow liquid (hereinafter referred to as an aldehyde compound 4) was prepared. The infrared absorption spectrum of the aldehyde compound 4 is illustrated in FIG. 11.

### (3) Synthesis of Methylol Compound 4

The procedure for synthesizing the methylol compound 4 is illustrated by the following reaction formula.

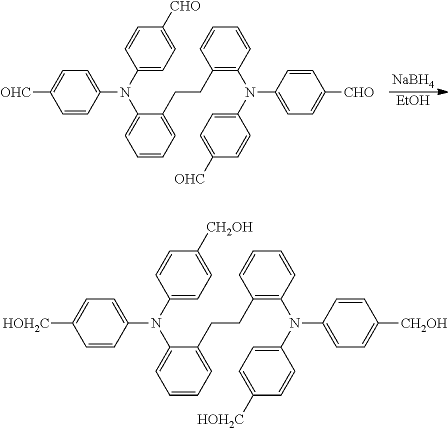

Specifically, the following components were fed into a four-necked flask.

| | | |
|---|---|---|
| Aldehyde compound 4 prepared above | 9.43 | g |
| Ethanol | 100 | ml |

The components were mixed by an agitator at room temperature. Next, 2.72 g of sodium borohydride was added there to while agitated, and the mixture was agitated for 7 hours. The reaction product was extracted using ethyl acetate, and the extracted material was subjected to a dehydration treatment using magnesium sulfate, followed by an absorption treatment using activated earth and silica gel, filtering, washing and condensing, resulting in preparation of an amorphous material. The amorphous material was dispersed in n-hexane, followed by filtering, washing and drying. Thus, 8.53 g of a white amorphous material (i.e., methylol compound 4) was prepared. The infrared absorption spectrum of the methylol compound 4 is illustrated in FIG. 12.

As mentioned above, by subjecting aldehyde compounds having formula (2) (e.g., aldehyde compounds 1-4), which have been synthesized by the synthesizing methods mentioned above for example, to a reduction reaction, methylol compounds having formula (1) (e.g., methylol compounds 1-4) can be prepared. By using the methods, the other methylol compounds 5 and 6 listed in Table 1 can also be prepared.

In order to evaluate the charge transportability of the methylol compounds 1-4, photoreceptors including a charge transport layer including the methylol compound 1, 2, 3 or 4 were prepared and evaluated in following Application Examples 1-4 while compared with comparative photoreceptors of Comparative Examples 1 and 2.

### Application Example 1

An undercoat layer coating liquid having the following formula was applied on an aluminum plate, and then dried to form an undercoat layer having a thickness of 0.3 μm on the aluminum plate.

| | |
|---|---|
| Polyamide resin | 2 parts |
| (CM-8000 from Toray Industries, Inc.) | |
| Methanol | 49 parts |
| Butanol | 49 parts |

Next, a charge generation layer coating liquid having the following formula was applied on the undercoat layer, and then dried to form a charge generation layer having a thickness of 0.3 μm on the undercoat layer.

| | |
|---|---|
| Bisazo pigment having the following formula | 2.5 parts |
| 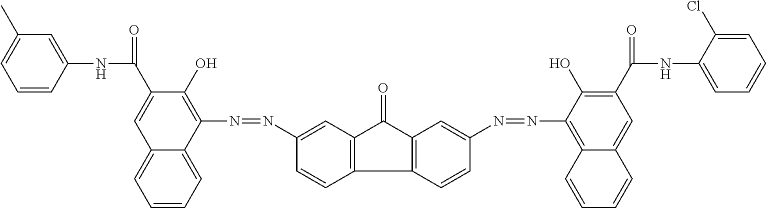 | |
| Polyvinyl butyral | 0.5 parts |
| (XYHL from Union Carbide Corp.) | |
| Cyclohexanone | 200 parts |
| Methyl ethyl ketone | 80 parts |

Further, a charge transport layer coating liquid having the following formula was coated on the charge generation layer, and then dried to form a charge transport layer having a thickness of 20 μm on the charge generation layer.

| | |
|---|---|
| Bisphenol Z-form polycarbonate | 10 parts |
| (PANLITE TS-2050 from Teijin Chemicals Ltd.) | |
| Methylol compound 1 prepared in Synthesis Example 1 | 10 parts |
| (Charge transport material) | |
| Tetrahydrofuran (THF) | 80 parts |
| 1% THF solution of a silicone oil | 0.2 parts |
| (Silicone oil: KF-50-100CS from Shin-Etsu Chemical | |
| Co., Ltd.) | |

Thus, a photoreceptor 1 was prepared.

### Application Example 2

The procedure for preparation of the photoreceptor in Application Example 1 was repeated except that the methylol compound 1 in the charge transport layer coating liquid was replaced with the methylol compound 2 prepared in Synthesis Example 2.

Thus, a photoreceptor 2 was prepared.

### Application Example 3

The procedure for preparation of the photoreceptor in

Application Example 1 was repeated except that the methylol compound 1 in the charge transport layer coating liquid was replaced with the methylol compound 3 prepared in Synthesis Example 3.

Thus, a photoreceptor 3 was prepared.

### Application Example 4

The procedure for preparation of the photoreceptor in Application Example 1 was repeated except that the methylol compound 1 in the charge transport layer coating liquid was replaced with the methylol compound 4 prepared in Synthesis Example 4.

Thus, a photoreceptor 4 was prepared.

### Comparative Application Example 1

The procedure for preparation of the photoreceptor in Application Example 1 was repeated except that the methylol compound 1 in the charge transport layer coating liquid was replaced with a comparative compound having the following formula (C1).

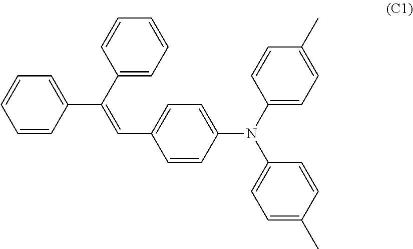

Thus, a photoreceptor 5 was prepared.

### Comparative Application Example 2

The procedure for preparation of the photoreceptor in Application Example 1 was repeated except that the methylol compound 1 in the charge transport layer coating liquid was replaced with a comparative methylol compound having the following formula (C2).

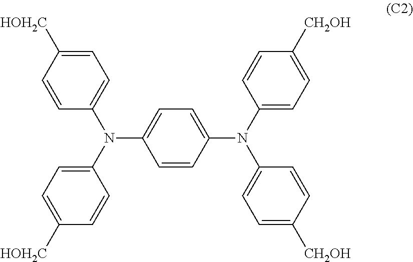

Thus, a photoreceptor 6 was prepared.

The charge transport property of each of the photoreceptors 1-6 prepared above was evaluated using an instrument, ELECTROSTATIC PAPER ANALYZER EPA-8200 from Kawaguchi Electric Works. Specifically, after a DC voltage of −6 kV was applied to each photoreceptor so that the photoreceptor has a potential of −800V, the charged photoreceptor was irradiated with light at a luminance of 4.5 lux using a tungsten lamp to determine a half potential time at which the potential (−800V) of the photoreceptor is decayed to −400V (i.e., to determine a half potential exposure E1/2 (in units of lux·sec)), and a residual potential of the photoreceptor determined when the light irradiation operation was performed for 30 seconds. In this regard, the smaller half potential exposure E1/2 a photoreceptor has, the better photosensitivity the photoreceptor has. In addition, the less residual potential a photoreceptor has, the smaller number of charge traps the photoreceptor has.

In addition, the solvent resistance of each photoreceptor was evaluated. Specifically, a portion of 10 m×10 mm of the surface of each photoreceptor was touched with a finger to adhere oils and fats discharged therefrom to the surface portion. After the photoreceptor was allowed to settle in a dark room under conditions of 45° C. and 43% RH, the surface portion was observed with a microscope to determine whether the portion has cracks. The solvent resistance property of the photoreceptors is graded as follows.
◯: No cracks (Good)
Δ: Four or less cracks are formed. (Acceptable)
×: Five or more cracks are formed (i.e., cracks are formed on the entire of the surface portion). (Unusable)

The evaluation results are shown in Table 2 below.

TABLE 2

| Photo- | | Half potential | Residual | |
|---|---|---|---|---|
| receptor | Charge transport | exposure E½ | potential | Solvent |
| No. | material | (lux · sec) | (V) | resistance |
| 1 | Methylol compound | 0.56 | 0 | ◯ |
| | 1 | | | |
| 2 | Methylol compound | 0.58 | 0 | ◯ |
| | 2 | | | |
| 3 | Methylol compound | 0.43 | 0 | ◯ |
| | 3 | | | |
| 4 | Methylol compound | 0.60 | 0 | ◯ |
| | 4 | | | |
| 5 | Comparative | 0.56 | 0 | X |
| | compound C1 | | | |
| 6 | Comparative | 3.87 | 45 | Δ |
| | methylol compound | | | |
| | C2 | | | |

It is apparent from Table 2 that the photoreceptors 1-4 which are examples of the photoreceptor of the present invention have as good half potential exposure (photosensitivity) and residual potential (charge trapping property) as the comparative photoreceptor (i.e., photoreceptor 5). Namely, the methylol compounds 1-4 have good charge transportability.

In addition, the examples of the photoreceptor of the present invention have good solvent resistance. The comparative photoreceptor (photoreceptor 5) has poor solvent resistance. Further, the other comparative photoreceptor (photoreceptor 6), which includes a methylol compound C2, has worse solvent resistance than the photoreceptor of the present invention. The reason therefor is considered to be that the methylol compound C2 has a rigid structure.

Next, in order to evaluate the crosslinking property of the methylol compounds 1-4, crosslinked layers were prepared using the methylol compounds 1-4 to measure the gel fraction thereof in comparison with comparative crosslinked layers of Comparative Examples 1 and 2.

### Example 1

The following components were mixed to prepare a coating liquid A.

| | |
|---|---|
| Methylol compound 1 prepared in Synthesis | 10 parts |
| Example 1 | |
| Toluene-2,4-diisocyanate (TDI) | 10 parts |
| (from Tokyo Chemical Industry Co., Ltd.) | |
| Tetrahydrofuran | 80 parts |

The coating liquid A was applied on an aluminum plate, and then dried to an extent such that the surface of the coated layer is not damaged even when the surface is touched with a finger, followed by heating for 30 minutes at 130° C. to form a crosslinked layer A having a thickness of 5 μm. The gel fraction of the crosslinked layer A was measured by the following method.
(1) The combination of the layer A and the aluminum plate is weighed to determine the weight (W1) of the layer A;
(2) The layer A is dipped in tetrahydrofuran at 25° C. for 5 days;
(3) After the dipping test, the combination of the layer A and the aluminum plate is naturally dried to remove tetrahydrofuran therefrom;
(4) The combination of the layer A and the aluminum plate is weighed to determine the weight (W2) of the layer A after the dipping treatment; and
(5) The gel fraction of the layerA is determined from the following equation:

Gel fraction (%)=(W2/W1)×100.

### Example 2

The procedure for preparation and evaluation of the layer A was repeated except that the methylol compound 1 in the coating liquid A was replaced with the methylol compound 2.

Thus, a crosslinked layer B was prepared, and the gel fraction thereof was measured.

### Example 3

The procedure for preparation and evaluation of the layer A was repeated except that the methylol compound 1 in the coating liquid A was replaced with the methylol compound 3.

Thus, a crosslinked layer C was prepared, and the gel fraction thereof was measured.

### Example 4

The procedure for preparation and evaluation of the layer A was repeated except that the methylol compound 1 in the coating liquid A was replaced with the methylol compound 4.

Thus, a crosslinked layer D was prepared, and the gel fraction thereof was measured.

### Comparative Example 1

The procedure for preparation and evaluation of the layer A was repeated except that the methylol compound 1 in the coating liquid A was replaced with a comparative charge transport compound having the following formula C3.

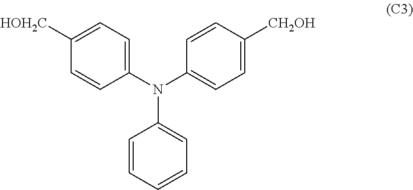

Thus, a crosslinked layer E was prepared, and the gel fraction thereof was measured.

### Comparative Example 2

The procedure for preparation and evaluation of the layer A was repeated except that the methylol compound 1 in the coating liquid A was replaced with a comparative charge transport compound having the following formula C4.

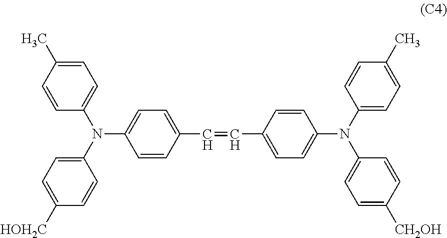

Thus, a crosslinked layer F was prepared, and the gel fraction thereof was measured.

The gel fraction of the layers A-F is shown in Table 3 below.

TABLE 3

| Layer | Gel fraction (%) |
|---|---|
| A (Crosslinked layer of | 99 |
| methylol compound 1) | |
| B (Crosslinked layer of | 98 |
| methylol compound 2) | |
| C (Crosslinked layer of | 98 |
| methylol compound 3) | |
| D (Crosslinked layer of | 99 |
| methylol compound 4) | |
| E (Crosslinked layer of | 90 |
| comparative compound C3) | |
| F (Crosslinked layer of | 88 |
| comparative compound C4) | |

It is apparent from Table 3 that each of the crosslinked layers formed by the methylol compounds 1-4 has a higher gel fraction than the crosslinked layers formed by the comparative charge transport compounds C3 and C4. Therefore, the methylol compounds can be preferably used as organic functional materials for various organic semiconductor devices because of being capable of forming layers having good resistance to abrasion and scratch, which is required for such organic semiconductor devices.

In this regard, the above-mentioned comparative compound C1, which has good charge transportability, cannot form a crosslinked layer.

As mentioned above, methylol compounds having formula (1) have a good charge transportability while being capable of forming a highly-crosslinked layer having good resistance to mechanical strength and heat whereas comparative compounds do not have a good combination of charge transportability and resistance to mechanical strength and heat. Therefore, the methylol compound of the present invention can be preferably used for various organic semiconductor devices such as electrophotographic photoreceptors.

Additional modifications and variations of the present invention are possible in light of the above teachings. It is therefore to be understood that within the scope of the appended claims the invention may be practiced other than as specifically described herein.

This document claims priority and contains subject matter related to Japanese Patent Application No. 2009-230220, filed on Oct. 2, 2009, the entire contents of which are herein incorporated by reference.

What is claimed is:

1. A methylol compound having the following formula (1): 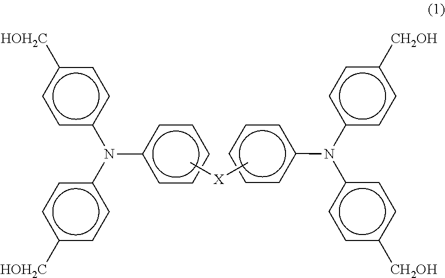
wherein X represents a group —O—, —CH₂—, —CH═CH—, or —CH₂CH₂—.

2. A method for preparing the methylol compound according to claim 1, comprising:
providing an aldehyde compound having the following formula (2): 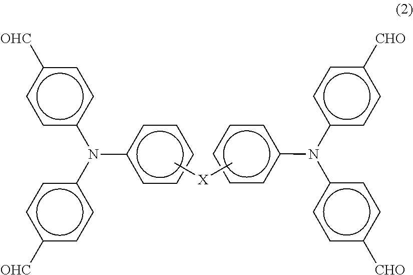
wherein X represents a group —O—, —CH₂—, —CH═CH—, or —CH₂CH₂—; and
subjecting the aldehyde compound to a reduction reaction in the presence of a reducing agent.

3. A charge transport layer comprising the methylol compound according to claim 1 and a binder resin.

4. An electrophotographic photoreceptor comprising:
a substrate;
an optional undercoat layer located on the substrate;
a charge generation layer; and
the charge transport layer according to claim 3,
wherein the charge generation layer and the charge transport layer are overlaid on the substrate or the optional undercoat layer.

5. An aldehyde compound having the following formula (2): 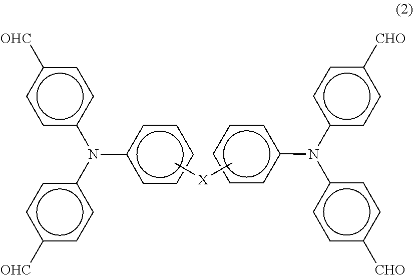
wherein X represents a group —O—, —CH₂—, —CH═CH—, or —CH₂CH₂—.
